(19) United States
(12) Patent Application Publication
(10) Pub. No.: US 2014/0132924 A1
(43) Pub. Date: May 15, 2014

(54) OPHTHALMIC APPARATUS AND ALIGNMENT DETERMINATION METHOD

(71) Applicant: CANON KABUSHIKI KAISHA, Tokyo (JP)

(72) Inventors: Osamu Sagano, Inagi-shi (JP); Daisuke Kawase, Ichikawa-shi (JP)

(73) Assignee: CANON KABUSHIKI KAISHA, Tokyo (JP)

(21) Appl. No.: 14/064,330

(22) Filed: Oct. 28, 2013

(30) Foreign Application Priority Data
Nov. 9, 2012 (JP) 2012-247753

(51) Int. Cl. A61B 3/15

(52) U.S. Cl. CPC A61B 3/152
USPC 351/208; 351/246

(57) ABSTRACT

An ophthalmic apparatus that determines whether alignment with a subject eye is complete using an observation image of an anterior ocular segment of the subject eye obtained through a split prism determines whether or not the alignment is a success or failure based on a position of a pupil image on respective lines of a line pair that are parallel to a boundary of the split prism and are equidistant from the boundary in the observation image. The ophthalmic apparatus determines the success or failure of alignment for a plurality of line pairs whose distances from the boundary are different, and determines whether or not the alignment is complete based on the obtained determination results.

### BACKGROUND OF THE INVENTION

1. Field of the Invention

The present invention relates to ophthalmic apparatuses used in ophthalmological clinics and the like, and to alignment determination methods therefor.

2. Description of the Related Art

Generally, when observing the anterior ocular segment of a subject eye using a fundus camera, an auxiliary lens optical system is inserted into the optical path and alignment including adjusting the imaging optical axis against the pupil, adjusting the working distance between an objective lens and the subject eye, and so on is carried out. The auxiliary lens optical system is retracted from the optical path when it has been determined that the alignment is complete, after which the fundus is observed, focused on, and imaged. Generally, when observing the anterior ocular segment, an image separating prism (split prism) that serves as the auxiliary lens optical system is inserted into the optical path of the observation optical system, and a user who is observing an anterior ocular segment image (an anterior ocular split image) determines the success or failure of alignment between the subject eye and the optical system of the apparatus (Japanese Patent Laid-Open No. 2003-245253). Imaging the anterior ocular segment while observing the anterior ocular segment and automatically detecting when alignment has been completed based on the resulting image signal has also been proposed.

However, when processing an image signal of the anterior ocular segment while observing the anterior ocular segment using a fundus camera as described above, there have been cases where the detection of the pupil or the detection that alignment is complete (that is, alignment determination) have failed due to the influence of reflected light resulting from the anterior ocular segment illumination. In this case, the fundus camera cannot automatically transit from an anterior ocular segment observation state to a fundus observation state, making it necessary for an operator to determine anterior ocular alignment him/herself and manually switch from the anterior ocular segment observation state to the fundus observation state. This has impeded the smooth operation of the fundus camera.

### SUMMARY OF THE INVENTION

An embodiment provides a fundus camera capable of determining, automatically and with certainty, whether anterior ocular segment alignment has been completed.

According to one aspect of the present invention, there is provided an alignment determination method for an ophthalmic apparatus that determines whether alignment with a subject eye is complete using an observation image of an anterior ocular segment of the subject eye obtained through a split prism, the method comprising: a judging step of determining the success or failure of alignment based on a position of a pupil image on respective lines in a line pair that are parallel to a boundary in the split prism and are equidistant from the boundary in the observation image; and a determining step of determining whether or not the alignment is complete based on a determination result obtained by executing the judging step for a plurality of line pairs having different distances from the boundary.

Furthermore, according to another aspect of the present invention, there is provided an alignment determination method for an ophthalmic apparatus that determines whether alignment with a subject eye is complete using an observation image of an anterior ocular segment of the subject eye obtained through a split prism, the method comprising: a detection step of detecting a reflection image resulting from anterior ocular segment illumination in the observation image; a setting step of setting a line pair whose lines are parallel to a boundary of the split prism and that are equidistant from the boundary so that the line pair does not overlap with the reflection image in the observation image; and a judging step of determining whether or not the alignment is complete based on positions, in the observation image, of the pupil image on the respective lines in the line pair set in the setting step.

Furthermore, according to another aspect of the present invention, there is provided an alignment determination method for an ophthalmic apparatus that determines whether alignment with a subject eye is complete using an observation image of an anterior ocular segment of the subject eye obtained through a split prism, the method comprising: a calculation step of calculating respective centers of gravity for two pupil areas of a pupil image in the observation image that have been separated by a boundary of the split prism; and a judging step of determining whether or not the alignment is complete based on the positions, in the observation image, of the two centers of gravity calculated in the calculation step and a position, in the observation image, of a split center.

Furthermore, according to another aspect of the present invention, there is provided an ophthalmic apparatus that determines whether alignment with a subject eye is complete using an observation image of an anterior ocular segment of the subject eye obtained through a split prism, the apparatus comprising: a judging unit configured to determine the success or failure of alignment based on a position of a pupil image on respective lines in a line pair that are parallel to a boundary in the split prism and are equidistant from the boundary in the observation image; and a determining unit configured to determine whether or not the alignment is complete based on a determination result from the judging unit for a plurality of line pairs having different distances from the boundary.

Furthermore, according to another aspect of the present invention, there is provided an ophthalmic apparatus that determines whether alignment with a subject eye is complete using an observation image of an anterior ocular segment of the subject eye obtained through a split prism, the apparatus comprising: a detection unit configured to detect a reflection image resulting from anterior ocular segment illumination in the observation image; a setting unit configured to set a line pair whose lines are parallel to a boundary of the split prism and that are equidistant from the boundary so that the line pair does not overlap with the reflection image in the observation image; and a judging unit configured to determine whether or not the alignment is complete based on positions, in the observation image, of the pupil image on the respective lines in the line pair set by the setting unit.

Furthermore, according to another aspect of the present invention, there is provided an ophthalmic apparatus that determines whether or not alignment with a subject eye is complete using an observation image of an anterior ocular segment of the subject eye obtained through a split prism, the apparatus comprising: a calculation unit configured to calculate respective centers of gravity for two pupil areas of a pupil image in the observation image that have been separated by a boundary of the split prism; and a judging unit configured to determine whether or not the alignment with the subject eye is complete based on the positions, in the observation image, of the two centers of gravity calculated by the calculation unit and a position, in the observation image, of a split center.

Further features of the present invention will become apparent from the following description of exemplary embodiments with reference to the attached drawings.

### BRIEF DESCRIPTION OF THE DRAWINGS

FIG. 1 is a diagram illustrating an example of the configuration of a fundus camera according to an embodiment.

FIG. 2 is a front view of an anterior ocular segment split prism.

FIG. 3 is a front view of a monitor in which an anterior ocular segment image is projected.

FIGS. 4A to 4D are diagrams illustrating pupil positions and alignment states during anterior ocular observation.

FIG. 5 is a flowchart illustrating an anterior ocular segment alignment determination process according to a first embodiment.

FIG. 6 is a flowchart illustrating an anterior ocular segment alignment determination process according to the first embodiment.

FIG. 7 is a diagram illustrating alignment determination according to the first embodiment.

FIG. 8 is a flowchart illustrating an anterior ocular segment alignment determination process according to a second embodiment.

FIGS. 9A and 9B are diagrams illustrating alignment determination according to the second embodiment.

FIG. 10 is a diagram illustrating alignment determination according to a third embodiment.

FIG. 11 is a flowchart illustrating an anterior ocular segment alignment determination process according to the third embodiment.

FIG. 12 is a flowchart illustrating an anterior ocular segment alignment determination process according to a fourth embodiment.

FIG. 13 is a flowchart illustrating an anterior ocular segment alignment determination process according to a fifth embodiment.

FIG. 14 is a diagram illustrating alignment determination according to the fifth embodiment.

### DESCRIPTION OF THE EMBODIMENTS

Embodiments of the present invention will be described in detail hereinafter. Although the following embodiments describe a fundus camera as an example, the present invention is not particularly limited thereto, and can be applied in any ophthalmic apparatus that determines whether alignment has been completed using an observation image of the anterior ocular segment of a subject eye obtained through a split prism. For example, the present invention is clearly applicable in ophthalmic imaging apparatuses/measurement devices such as OCT (optical coherence tomography) apparatuses, tonometers, and the like.

### First Embodiment

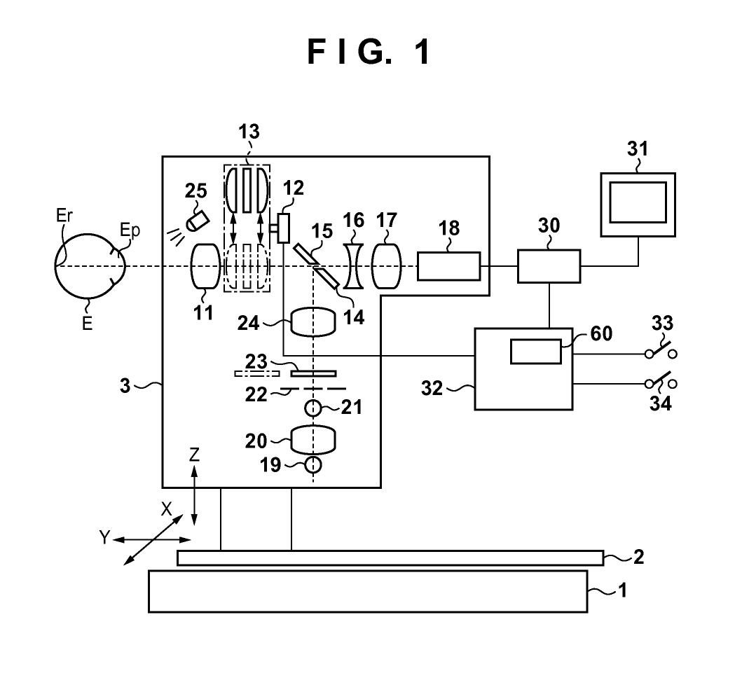

FIG. 1 is a diagram illustrating an example of the configuration of a fundus camera according to a first embodiment. A horizontal section mobile platform 2 capable of moving forward-backward and left-right (X and Y directions) is installed upon a base 1, and an optical system body 3 is provided on the horizontal section mobile platform 2 so as to be capable of moving up-down (a Z direction).

In the optical system body 3, an auxiliary lens optical system 13 that can be moved in and out of an optical path by a driving unit 12 and a perforated mirror 14 are disposed upon an optical axis of an objective lens 11 that opposes a subject eye E. Furthermore, an imaging aperture 15 provided in a hole of the perforated mirror 14, a focusing lens 16 capable of moving along the optical axis, an imaging lens 17, and an imaging unit 18 are disposed on the optical axis of the objective lens 11.

An observation light source 19, a condenser lens 20, an imaging light source 21 that emits a flash, an aperture 22 having a ring-shaped opening, an infrared light cutting filter 23 that is disposed so as to be insertable/retractable and that blocks infrared light, and a relay lens 24 are disposed in an optical path of an illumination optical system that illuminates the subject eye E. An imaging optical system is configured by the array of these optical members, from the observation light source 19 that emits fixed infrared light, to the perforated mirror 14. Furthermore, an infrared light source 25 for illuminating the anterior ocular segment of the subject eye is provided in the vicinity of the objective lens 11, and an anterior ocular segment illumination unit is configured as a result. Note that the infrared light source 25 is configured of, for example, an infrared LED or the like that emits infrared light.

An output of the imaging unit 18 is connected to an image control unit 30 having functions for storing image data, performing computation control, and so on. An output of the image control unit 30 is connected to a monitor 31, and furthermore, an output of an operation/display control unit 32 is connected to the image control unit 30. The operation/display control unit 32 includes an alignment determination unit 60 for automatically determining the success or failure of alignment with the subject eye based on an observation image obtained from the imaging unit 18. In the case where the alignment determination unit 60 has determined that the alignment is a success, the operation/display control unit 32 recognizes that the alignment is complete. The operation/display control unit 32 uses the driving unit 12 to cause the auxiliary lens optical system 13 to retract from the optical axis of the objective lens 11, and automatically changes the observation state from an anterior ocular segment observation state to a fundus observation state. It is assumed that the operation/display control unit 32 includes a CPU (not shown) and the alignment determination unit 60 is implemented by the CPU executing a predetermined program; however, the embodiment is not limited thereto, and the configuration may instead employ a FPGA, for example. An imaging switch 33 that causes the imaging light source 21 to emit light via a light emission control unit (not shown), an anterior ocular/fundus toggle switch 34, and the driving unit 12 are connected to the operation/display control unit 32.

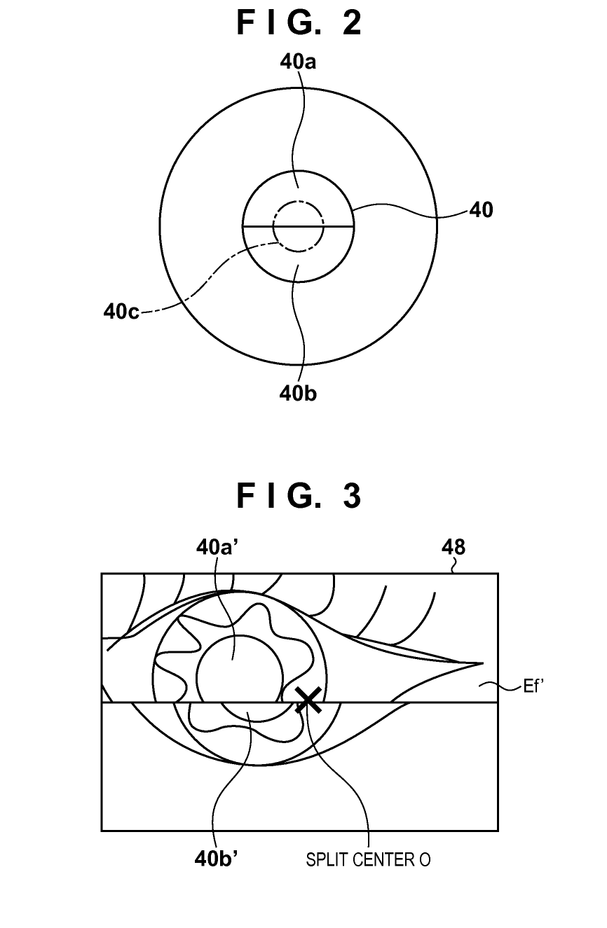

As shown in FIG. 2, the auxiliary lens optical system 13 is provided with a split prism 40 having a slope that differs between an upper half 40a and a lower half 40b of a central area, and the split prism 40 is configured to deflect left and right light rays separately. An alignment mark 40c is formed on a back surface of the split prism 40. During imaging, an operator sits a subject in front of the fundus camera and first roughly positions (aligns) the subject eye E and the fundus camera while observing the anterior ocular segment using infrared light. Thus immediately after the power is turned on, the operation/display control unit 32 automatically drives the driving unit 12 so as to insert the auxiliary lens optical system 13 into the optical path, and furthermore turns on the infrared light source 25 for illuminating the anterior ocular segment. This sets the fundus camera to the anterior ocular segment observation state.

Next, using an operation unit (not shown), the operator adjusts the focusing lens 16 so that the alignment mark 40c on the prism 40 is maximally focused in the monitor 31. Here, the usability can be further increased by configuring the focusing lens 16 to automatically move to a predetermined position in the anterior ocular segment observation state.

In the anterior ocular segment observation state, the infrared light cutting filter 23 is retracted outside of the optical path. FIG. 3 illustrates an anterior ocular segment observation image Ef′ captured by the imaging unit 18. In the case where the working distance between the subject eye and the optical system is incorrect due to effects of the prism 40, an image 40a′ of the upper half of the pupil and an image 40b′ of the lower half of the pupil will be skewed horizontally. The operator then aligns the subject eye with the fundus camera in the vertical and horizontal directions so that the center of the pupil matches a split center O. Although the present embodiment describes an example in which the actual alignment operation is performed by the operator, it should be noted that the invention is not limited thereto. For example, the fundus camera may automatically carry out the alignment by processing the anterior ocular segment observation image obtained by the imaging unit 18.

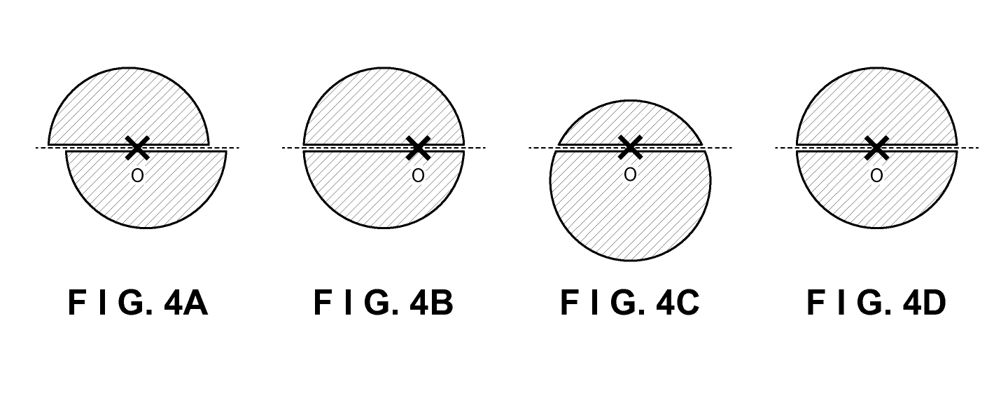

FIGS. 4A to 4D are diagrams illustrating pupil positions and alignment states, and illustrate only the pupil area shown in FIG. 3. From FIG. 4A, it can be seen that the upper and lower pupil areas are skewed horizontally and that the adjustment of the working distance is insufficient. In FIG. 4B, the working distance is correct, but the center of the pupil is skewed to the left from the split center O. In FIG. 4C, the working distance and horizontal alignment are correct, but the center of the pupil is skewed downward from the split center O. FIG. 4D illustrates a state in which the center of the pupil is in the split center and no skew is caused by the prism 40, and thus the alignment is correct (that is, the alignment is complete).

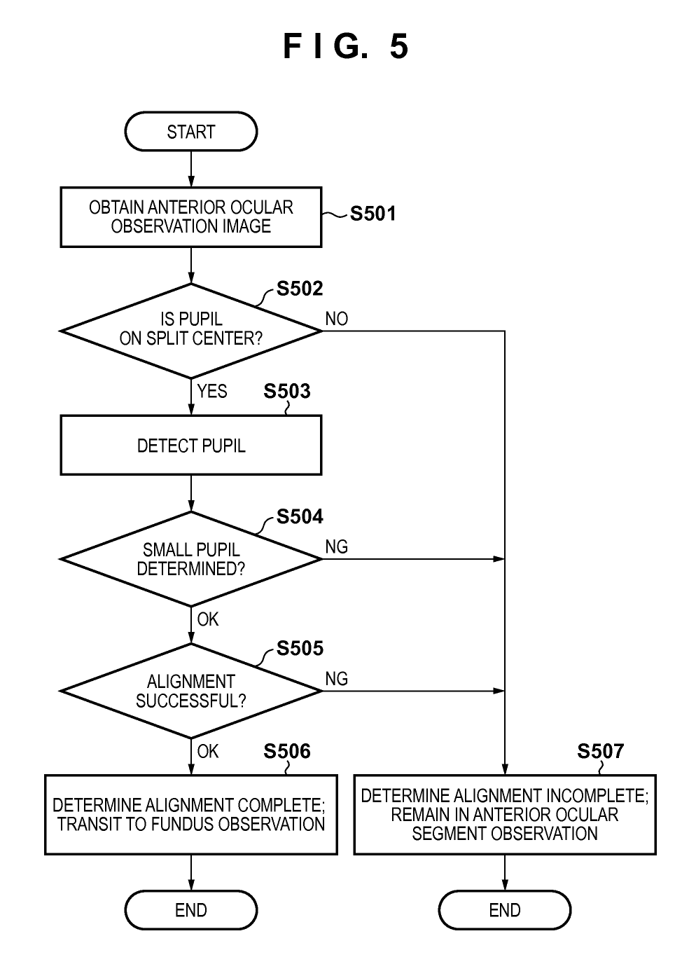

The alignment determination unit 60 determines the success or failure of the anterior ocular segment alignment for the anterior ocular segment observation image Ef′ by analyzing an anterior ocular split image, which is an observation image captured by the imaging unit 18 via the prism 40. Next, a method for determining the success or failure of the anterior ocular segment alignment performed by the alignment determination unit 60 will be described. FIG. 5 is a flowchart illustrating an anterior ocular segment alignment determination method according to the present embodiment.

The alignment determination unit 60 obtains an observation image of the anterior ocular segment from the imaging unit 18 (S501), and determines whether or not the center of the pupil is in the split center (or is within a predetermined range from the split center) (S502). In the case where it is determined that the center of the pupil is not in the split center, the process is ended assuming the determination has failed (NO in S502; S507). On the other hand, in the case where it is determined that the center of the pupil is in the split center, the alignment determination unit 60 detects the pupil from the observation image (YES in S502; S503). When detecting the pupil, the alignment determination unit 60 binarizes the observation image. Although the binarization may be carried out using a predetermined threshold, image information such as an average value, a histogram, or the like of the observation image may be calculated and a threshold for binarization may then be set based on that information.

Next, the size of the pupil detected from the binarized observation image is determined, and it is determined whether or not the pupil in the observation image is a small pupil (S504). In the case where the size of the pupil in the observation image is smaller than a predetermined size, the processing ends assuming that the determination has failed (NG in S504; S507). Although this determination can be realized by calculating the surface area of the binarized pupil area, the determination may be carried out as follows, for example. First, the following are found:
(1) a maximum value of the horizontal width of the pupil in the upper split image;
(2) the height of the pupil from the split center in the upper split image;
(3) a maximum value of the horizontal width of the pupil in the lower split image; and
(4) the height of the pupil from the split center in the lower split image.

It is then determined whether either of (1) and (3), and the sum of (2) and (4), are greater than a given pupil diameter. In the case where the size of the pupil is smaller than that given pupil diameter, the processing ends assuming that the determination has failed (NG in S504; S507). Note that the maximum values, the heights, and the pupil diameter may be expressed as, for example, pixel values.

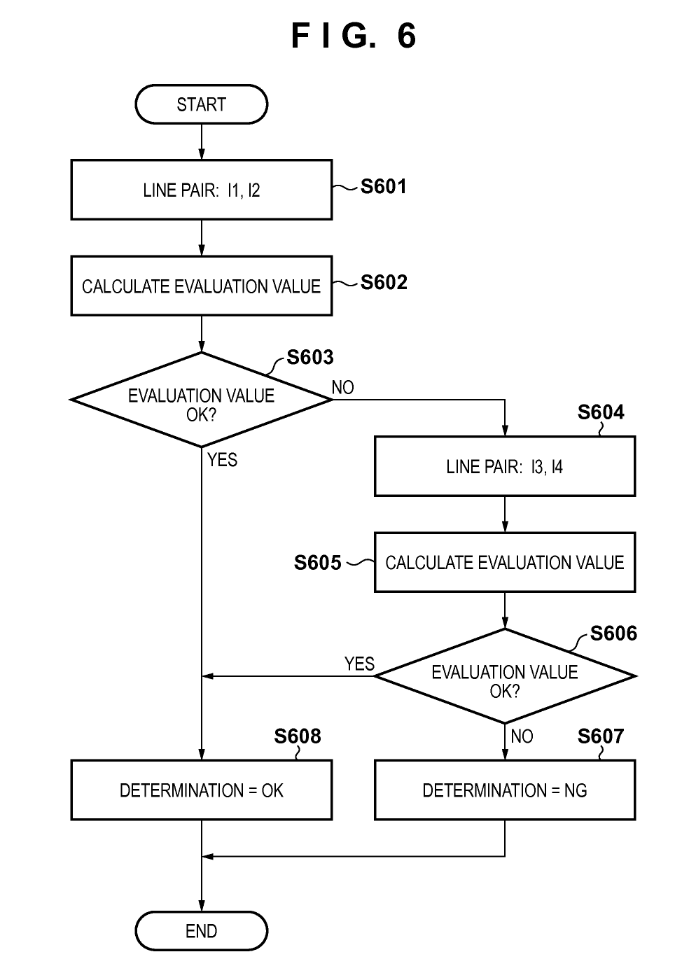

In the case where the size of the pupil in the observation image (the split image) is greater than or equal to a predetermined size, the alignment determination unit 60 performs alignment determination (OK in S504; S505). Next, the alignment determination carried out in S505 will be described in detail with reference to the flowchart in FIG. 6.

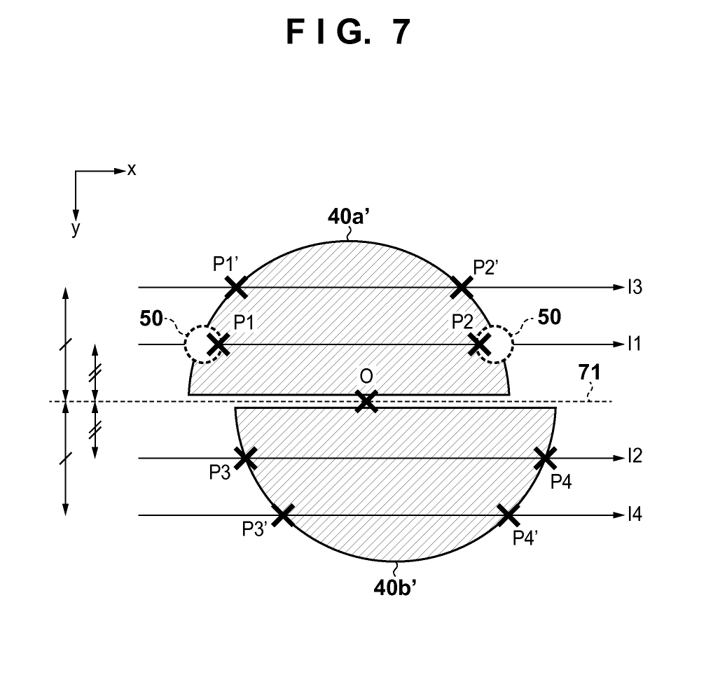

As shown in FIG. 7, in the alignment determination according to the first embodiment, line pairs [11, 12] and [13, 14] that are parallel to a boundary of the split prism (an anterior ocular split position 71) and whose respective lines are equidistant from the boundary are set in the anterior ocular segment observation image. Note that the distances of the line pair [11, 12] from the boundary is different from the distance of the line pair [13, 14] from the boundary, and the line pair [11, 12] is closer to the boundary than the line pair [13, 14]. The success or failure of the alignment is determined based on the position of the pupil image in each line of the line pairs. Note that the position of the pupil image in a line is detected based on the position of an edge of the pupil image in that line, as will be described in detail below. Next, the determination as to the success or failure of the alignment based on a pupil edge position in the line pairs will be described with reference to the flowchart in FIG. 6.

First, the alignment determination unit 60 sets the line pair [11, 12] as indicated in FIG. 7 (S601). Next, the alignment determination unit 60 detects an edge of the pupil on the lines from binarized image data cut out for each line in the line pair, and calculates evaluation values based on the detected positions (S602). More specifically, this is carried out as follows.

As shown in FIG. 7, the edge positions of the pupil on the line 11 are taken as P1 and P2, and the edge positions of the pupil on the line 12 are taken as P3 and P4. Here, the coordinates of a point Pi are taken as (xi,yi). Furthermore, the split center, which is the center position of the anterior ocular split image, is set to a point O (xo,yo) by design. The evaluation values are calculated through the following Formulas 1 to 6 based on the edge position of the pupil on the lines l1 and l2.

pupil length (lower): a1=x4−x3 (Formula 1)

skew amount 1 in horizontal direction: a2=x3−x1 (Formula 2)

skew amount 2 in horizontal direction: a3=x4−x2 (Formula 3)

pupil length (upper): a4=x2−x1 (Formula 4)

skew amount from center: a5=xo−(x1+x2)/2 (Formula 5)

skew amount from center: a6=(x3+x4)/2−xo (Formula 6)

Next, the alignment determination unit 60 determines whether or not all of the conditions indicated by the following Formulas 7 to 11 are met using the evaluation values a1 to a6 (S603), and determines that the alignment is successful in the case where all the conditions are met (YES in S603; S608).

−1≦a2≦+1 [pixel] (Formula 7)

−1≦a3≦+1 [pixel] (Formula 8)

−1≦a5≦+1 [pixel] (Formula 9)

−1≦a6≦+1 [pixel] (Formula 10)

−1≦a4−a1≦+1 [pixel] (Formula 11)

On the other hand, in the case where any of the conditions indicated by Formulas 7 to 11 are not met, the determination result indicates a failure, and the alignment determination unit 60 performs the same evaluation value calculation and determination process using the other line pair [13, 14] (NO in S603; S604, S605, S606). In the case where all of the conditions indicated by Formulas 7 to 11 are then met, it is determined that the alignment is a success (YES in S606; S608). On the other hand, in the case where any of the conditions indicated by Formulas 7 to 11 using the line pair [13, 14] have not been met (that is, in the case of a failure), the alignment determination unit 60 determines that the alignment is incorrect (NO in S606; at S607). Note that in the present embodiment, in the case where the determination for the line pair [11, 12] has failed, the determination is carried out once again using the line pair [13, 14]. As a result, even if, for example, a reflection image resulting from the anterior ocular segment illumination is present on the line pair [11, 12] and the edge positions P1 and P2 of the pupil area are erroneously detected, the determination is then carried out using the other line pair, making it possible to perform the determination without being affected by the anterior ocular segment illumination.

It is desirable for the interval between adjacent line pairs, or in other words, the distance between 11 and 13 and the distance between 12 and 14, to be slightly greater than an estimated spot size (that is, the size of the reflected image) in the case where the anterior ocular segment illumination appears in the image. Doing so makes it possible for one of the line pair [11, 12] and the line pair [13, 14] to avoid being influenced by the reflective image, and thus it is only necessary to set two line pairs. However, if the distance from the boundary of the split prism to the line pairs is too great, the pupil will take on a circular shape, and the positions at which P1 to P4 are detected will be extremely susceptible to variations due to the curvature factor thereof. Accordingly, it is preferable for the distance from the boundary of the split prism to the line pairs to be no greater than necessary.

Although the above describes an example in which two line pairs are set, three or more line pairs may be set as well. In this case, although the interval between adjacent line pairs can be set to be smaller than the size of the reflected image, at least the interval between the line pair that is closest to the boundary and the line pair that is furthest from the boundary is set to be greater than the size of the reflected image. Furthermore, in this case, it is preferable, in light of the aforementioned influence of the pupil curvature factor, to determine the success or failure of alignment starting with the line pair that is closest to the boundary of the split prism. The success or failure of alignment is determined in sequence using the set line pairs, and it is determined that alignment is complete when a determination result indicating success has been obtained; thus the determination is not performed for the remaining line pairs. The alignment is determined to be incomplete in the case where a determination result indicating success is not obtained even after the success or failure of alignment has been determined using all of the line pairs.

Although in the present embodiment, the determination is carried out using the second line pair in the case where the determination carried out using the first line pair indicates a failure, evaluation values may be calculated for both line pairs and the determination may be carried out using those evaluation values. The number of line pairs is not limited to two in this case as well, and evaluation values may be calculated using three or more line pairs, and the alignment determination may be carried out based thereon. The operation/display control unit 32 determines that alignment is complete in the case where any one of the determinations of the success or failure of alignment has indicated a successful alignment. Conversely, the operation/display control unit 32 determines that alignment is incomplete in the case where all of the determination results have indicated failure.

In the flowchart illustrated in FIG. 5, when the alignment determination result indicates a success, the operation/display control unit 32 determines that alignment is complete and switches the observation state from the anterior ocular segment to the fundus (OK in S505; S506). More specifically, the driving unit 12 retracts the auxiliary lens optical system 13 from the optical path and transits the fundus camera to the fundus observation state. On the other hand, in the case where the alignment determination result indicates a failure, the operation/display control unit 32 determines that alignment is incomplete, and maintains the anterior ocular segment observation state (NG in S505; S507).

Note that in the fundus observation state (S506), the operation/display control unit 32 extinguishes the infrared light source 25 for anterior ocular segment illumination and turns on the observation light source 19 that emits infrared light for fundus illumination. At this time, the infrared light cutting filter 23 is retracted outside of the optical path. The infrared light emitted by the observation light source 19 is focused by the condenser lens 20, traverses the imaging light source 21 and the opening of the aperture 22 that has a ring-shaped opening, passes through the relay lens 24, and is reflected to the left by the peripheral mirror portion of the perforated mirror 14. The infrared light reflected by the perforated mirror 14 passes through the objective lens 11 and a pupil Ep of the subject eye E, and illuminates a fundus Er.

An image of the fundus illuminated by infrared light in this manner once again passes through the objective lens 11, the imaging aperture 15, the focusing lens 16, and the imaging lens 17, is formed on the imaging unit 18, and is converted into an electrical signal. This signal is then inputted into the image control unit 30 and displayed in the monitor 31. The operator then performs focusing operations and confirms an imaging range by moving the focusing lens 16 using the operation unit (not shown) while viewing the image displayed in the monitor 31, and then manipulates the imaging switch 33 if the focus and imaging range are correct. The fundus imaging is carried out in this manner.

Having detected the input from the imaging switch 33, the operation/display control unit 32 inserts the infrared light cutting filter 23 into the optical path and causes the imaging light source 21 to emit light. The light emitted from the imaging light source 21 traverses the opening of the aperture 22, after which only visible light is allowed to pass by the infrared light cutting filter 23; this light passes through the relay lens 24 and is reflected to the left by the peripheral mirror portion of the perforated mirror 14. The visible light reflected by the perforated mirror 14 passes through the objective lens 11 and the pupil Ep and illuminates the fundus Er. An image of the fundus illuminated in this manner once again passes through the objective lens 11, the imaging aperture 15, the focusing lens 16, and the imaging lens 17, is formed on the imaging unit 18 and converted into an electrical signal, and is displayed in the monitor 31.

According to the first embodiment as described above, the alignment determination will not fail even in the case where the anterior ocular segment illumination appears in the anterior ocular segment observation image, and thus the determination can be carried out with certainty. In addition to enabling the alignment determination to be carried out accurately, providing the alignment determination unit 60 achieves a further effect of greatly improving the operability.

### Second Embodiment

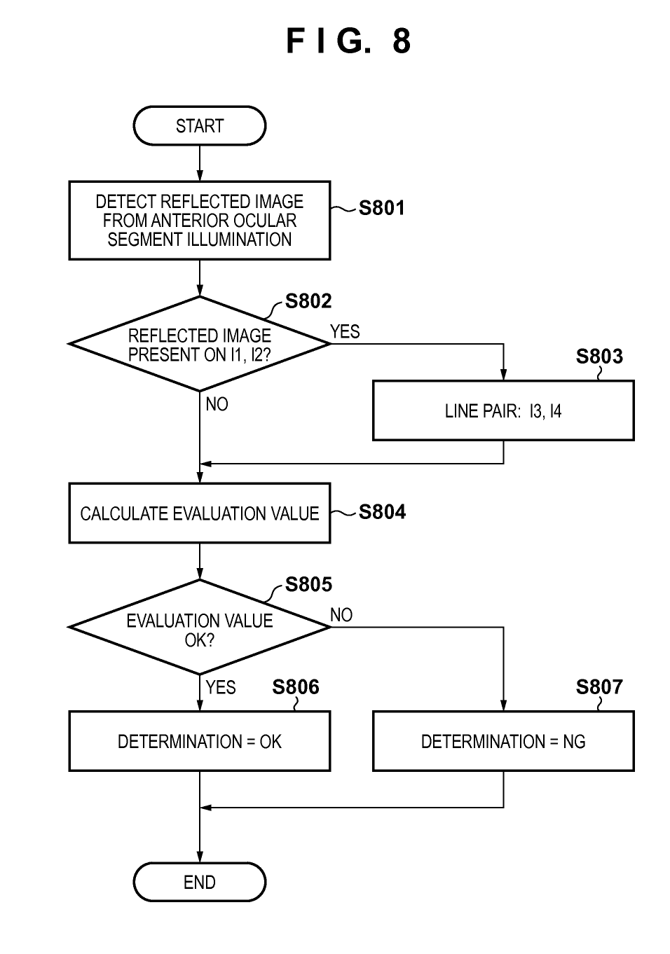

FIG. 8 is a flowchart illustrating operations performed by the alignment determination unit 60 according to a second embodiment. In the second embodiment, after the anterior ocular segment observation image is obtained, whether or not a bright point of reflection is present in the anterior ocular segment illumination is detected; then, in the case where anterior ocular segment illumination is present on a horizontal line pair, the determination is carried out after changing the position of the horizontal line pair. Descriptions will be given below using the flowchart in FIG. 8.

First, the anterior ocular segment observation image obtained in 5501 (the anterior ocular segment observation image prior to the binarization of S503) is obtained. Next, the image is binarized in order to detect a reflection image resulting from the anterior ocular segment illumination (S801). The anterior ocular segment illumination area is an area of extremely high brightness, and is often saturated to a maximum value in terms of image data. Accordingly, the reflection image resulting from the anterior ocular segment illumination can be detected if the binarization is carried out with a value greater than a given threshold. In the present embodiment, the anterior ocular segment observation image is binarized assuming that, for example, an area in an 8-bit image signal (having a maximum value of 255) whose brightness value is greater than 240 corresponds to anterior ocular segment illumination, and the threshold for the binarization performed in S801 is thus 240.

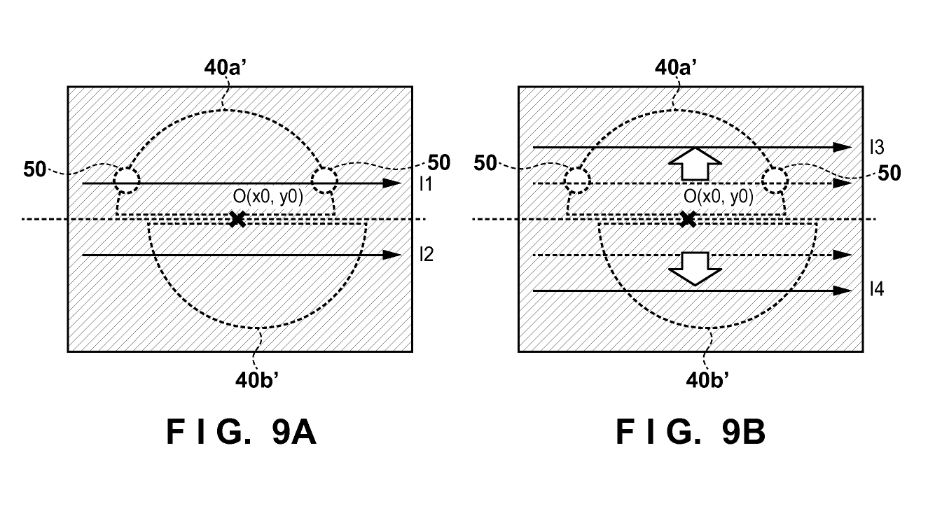

Next, the alignment determination unit 60 determines whether or not the reflection image resulting from the anterior ocular segment illumination is present on at least one of the lines in the line pair [11, 12]. FIGS. 9A and 9B schematically illustrate this state. In the case where the reflection image is not present on either of the lines 11 and 12, the success or failure of alignment is determined using the line pair [11, 12] (NO in S802; S804). Note that the determination formulas used at this time are the same as in the first embodiment, and the image used to detect the pupil edge positions is the binary image obtained in S503.

On the other hand, as shown in FIG. 9A, in the case where the reflection image resulting from the anterior ocular segment illumination is present on the line 11, the success or failure of alignment is not determined using the line pair [11, 12]; instead, the success or failure of alignment is determined using the line pair [13, 14] on which the reflection image resulting from the anterior ocular segment illumination is not present, as indicated in FIG. 9B (YES in S802; S803). Here, if the interval between the line pair [11, 12] and the line pair [13, 14] is set to be greater than the estimated size of the image resulting from the reflection, the image resulting from the reflection will not be present on at least one of the two line pairs, and thus it is sufficient to prepare two line pairs as described above.

It goes without saying that three or more line pairs can be used in the second embodiment, in the same manner as in the first embodiment. Likewise, in the case where three or more line pairs are used, the line pairs should be selected in order from the line pair that is closest to the boundary of the split prism in light of the influence of the pupil image curvature factor, in the same manner as in the first embodiment.

Furthermore, rather than selecting a line pair that avoids the image resulting from the reflection from a plurality of line pairs, a line pair may be set at a position that avoids a region containing the image resulting from the reflection and the success or failure of alignment may then be determined using the set line pair.

As described above, according to the second embodiment, erroneous determinations caused by reflections from the anterior ocular segment illumination appearing in the anterior ocular segment observation image can be prevented, and the alignment determination can be carried out with certainty.

### Third Embodiment

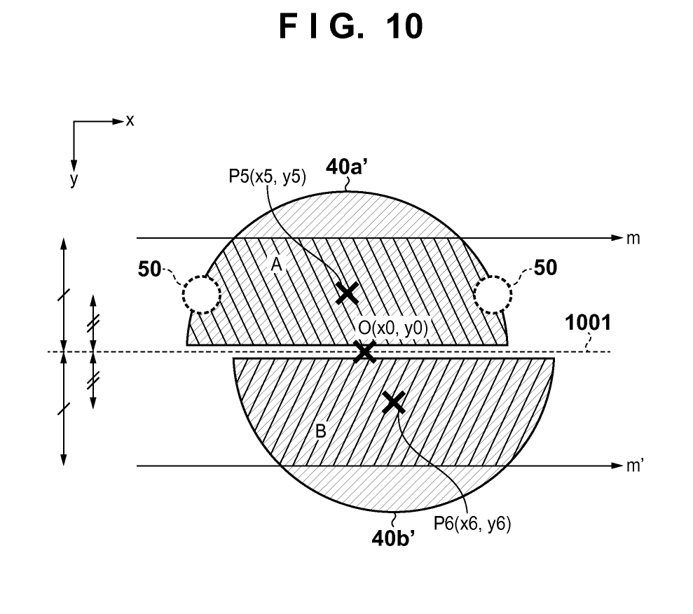

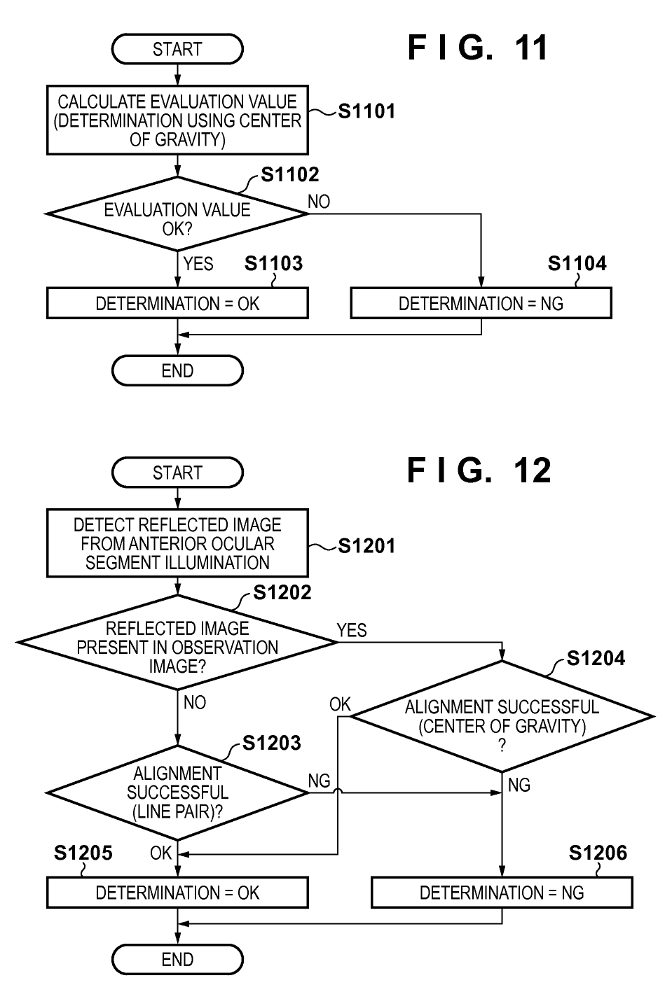

In the above first and second embodiments, the success or failure of alignment is determined by detecting the pupil position in the observation image based on the pupil edge positions in a line pair; however, in the third embodiment, the success or failure of alignment is determined by finding the surface area of part of the pupil and calculating the center of gravity thereof. FIG. 10 is a diagram illustrating the calculation of the center of gravity according to the third embodiment, and FIG. 11 is a flowchart illustrating a process by which the alignment determination unit 60 determines the success or failure of alignment according to the third embodiment.

The alignment determination unit 60 calculates evaluation values by calculating centers of gravity for portions A and B of a pupil image in the split image (the observation image) as shown in FIG. 10, in the area corresponding to the pupil in the binary image obtained through the binarization performed in S503 (S1101). The portion A and the portion B are set as follows. First, a line pair (a line m and a line m′) in which the lines are parallel to a split boundary line 1001 (the boundary of the split prism) and are the same distance from the split boundary line 1001 are set. The portion A corresponds to the surface area of the pupil surrounded by the split boundary line 1001 and the line m, whereas the portion B corresponds to the surface area of the pupil surrounded by the split boundary line 1001 and the line m′.

The alignment determination unit 60 then calculates evaluation values using the following Formulas 15 to 17, using a center of gravity P5 (x5,y5) of the portion A, a center of gravity P6 (x6,y6) of the portion B, and the split center O (xo,yo), and then determines the success or failure of alignment using the determination formulas indicated by Formulas 18 to 20.

depth direction determination formula: a7=x6−x5 (Formula 15)

vertical direction determination formula: a8=(y5+y6)/2−yo (Formula 16)

horizontal direction determination formula: a9(x5+x6)/2−xo (Formula 17)

Then, the alignment determination unit 60 determines whether or not the above evaluation values a7 to a9 meet all of the conditions indicated by the following Formulas 18 to 20 (S1102). In the case where the evaluation values a7 to a9 meet all of the conditions indicated in the Formulas 18 to 20, it is determined that the alignment is successful (YES in S1102; S1103), whereas in the case where the conditions are not met, it is determined that the alignment is unsuccessful (NO in S1102; S1104).

−1≦a7≦+1 [unit: pixel] (Formula 18)

−1≦a8≦+1 [unit: pixel] (Formula 19)

−1≦a9≦+1 [unit: pixel] (Formula 20)

By determining the success or failure of alignment through calculations that use a center of gravity in this manner, errors can be reduced by setting the size for calculating the surface area of the pupil portion to be sufficiently greater than the spot size (that is, the size of the reflection image resulting from the anterior ocular segment illumination), even in the case where, for example, the anterior ocular segment illumination appears in the portion A and the portion B. Furthermore, the reflection image resulting from the anterior ocular segment illumination may also be detected as in the second embodiment, and the parallel line pair m, m′ may be set so that the image resulting from the reflection is not present in the portion A and the portion B. The precision can be improved by setting the parallel line pair m, m′ so that the image resulting from the reflection is not present.

Although the determination is carried out based on the surface area of the pupil in a region enclosed by the split boundary line 1001 and the line m and a region enclosed by the split boundary line 1001 and the line m′ in the third embodiment, the surface area of the entirety of the pupil portions located above and below the split boundary line 1001 may be used instead. However, depending on the subject eye, the upper area of the pupil may be covered by the eyelid, and it is thus possible that the surface area cannot be correctly calculated. Accordingly, the desired effects can be achieved by setting the portion A and the portion B using the parallel line pair m, m′, and excluding positions in the upper area of the pupil that may be covered by the eyelid and corresponding positions in the lower area of the pupil.

According to the third embodiment as described above, the determination can be carried out with certainty and without failure even when anterior ocular segment illumination appears in the anterior ocular segment observation image.

### Fourth Embodiment

A difference between the fourth embodiment and the above first to third embodiments is that in the alignment determination, the reflection image resulting from the anterior ocular segment illumination is detected, and the determination is carried out based on the center of gravity in the case where the reflection image has been detected, whereas the determination is carried out based on horizontal lines in the case where the reflection image has not been detected. Here, the center of gravity determination corresponds to the alignment determination described in the third embodiment, whereas the horizontal line determination corresponds to the alignment determination described in the first or the second embodiment. Doing so makes it possible to avoid relying solely on the processing-intensive center of gravity determination, which in turn can reduce the load on the alignment determination unit 60 (the CPU) that carries out the calculations.

Hereinafter, determining the success or failure of alignment according to the fourth embodiment will be described using the flowchart shown in FIG. 12. The flowchart in FIG. 12 illustrates a process for determining the success or failure executed in the alignment determination process of S505 shown in FIG. 5. Accordingly, the processes of S501 to S504 (the determination as to whether the pupil is in the center and the small pupil determination) are executed before executing the alignment determination process. In the determination as to whether or not the pupil is in the center (S502), the processes that follow thereafter can be canceled in the case where there is a high level of misalignment and the pupil is not in the center, which can reduce the load on the CPU. Furthermore, in the case where the pupil is extremely small, there are cases where the evaluation values will have a high amount of error, the influence of the anterior ocular segment illumination will be great, and so on. Accordingly, in the case where a small pupil has been determined, there are situations where it is not preferable to automatically switch to fundus observation, situations where it is preferable to avoid such an automatic switch in order to make the operator aware of the small pupil, and so on; therefore, the alignment determination is not executed (S504). The alignment determination unit 60 detects the anterior ocular segment illumination from the anterior ocular segment observation image (S1201). Note that the anterior ocular segment illumination detection is as described in S801. The alignment determination unit 60 then determines whether or not a reflection image resulting from the anterior ocular segment illumination is present in the observation image (S1202). In the case where the reflection image resulting from the anterior ocular segment illumination is present in the observation image, the center of gravity determination described in the third embodiment is carried out (YES in S1202; S1204). On the other hand, in the case where the reflection image resulting from the anterior ocular segment illumination has not been detected in the observation image, the alignment determination is carried out using horizontal lines as described in the first embodiment (NO in S1202; S1203).

Note that in the present embodiment, the single line pair [11, 12] may be prepared. In the case where the alignment determination executed in S1203 or S1204 indicates that the alignment is successful, an alignment determination result indicating the success is obtained, and the processing advances to S506 (OK in S1203 or OK in S1204; S1205). On the other hand, in the case where the executed alignment determination indicates that the alignment is not successful, a determination result indicating failure is obtained and the processing advances to S507 (NG in S1203 or NG in S1204; S1206). Although whether or not a reflection image is present in the observation image is determined in S1202, it may instead be determined whether or not a reflection image is present on the line pair. In this case, the alignment determination is carried out using the line pair in the case where a reflection image is not present on the line pair, whereas the alignment determination is carried out using the center of gravity in the case where a reflection image is present on the line pair.

According to the fourth embodiment as described above, the alignment determination can be carried out with certainty and without failure even when anterior ocular segment illumination appears in the anterior ocular segment observation image.

### Fifth Embodiment

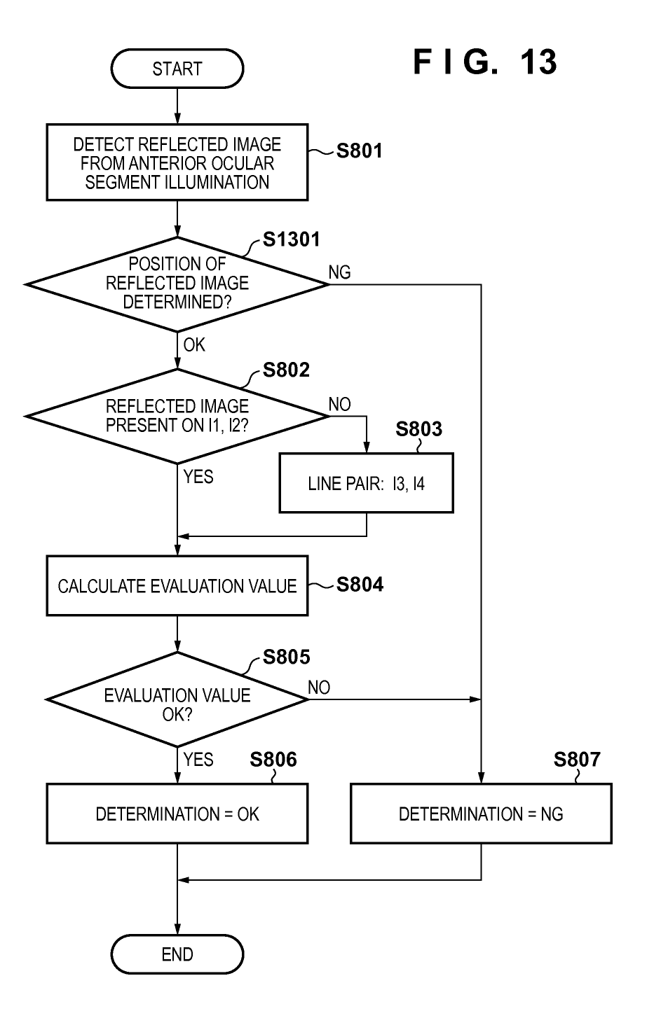

An alignment determination method performed by the alignment determination unit 60 according to the fifth embodiment adds improvements to the alignment determination method described in the second embodiment. Generally, fundus cameras are designed so that a position in the x direction where a reflection image resulting from the anterior ocular segment illumination appears is symmetrical relative to the x coordinate of the split center (that is, x0); in light of this, a determination based on the position of the reflection image resulting from anterior ocular segment illumination and the position of the split center is added in the present embodiment. Hereinafter, descriptions will be provided with reference to the flowchart in FIG. 13.

FIG. 13 is a flowchart, corresponding to FIG. 8 used in the second embodiment, that illustrates the alignment determination according to the present embodiment; here, processes that are the same as those in FIG. 8 are given the same step numbers as those in FIG. 8. In FIG. 13, after binarization for detecting a reflection image resulting from anterior ocular illumination and the process for detecting anterior ocular illumination (S801) are carried out, a process for determining whether the positions of two reflection images resulting from the anterior ocular illumination are symmetrical or asymmetrical (S1301) relative to the split center is added.

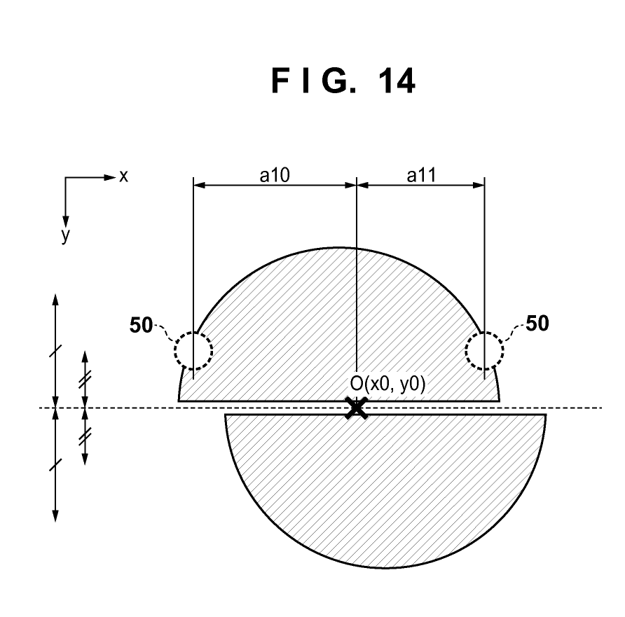

The determination of the anterior ocular segment illumination position in S1301 is carried out as indicated in FIG. 14. First, the alignment determination unit 60 finds evaluation values a10 and a11, indicated in FIG. 14, based on a positional relationship between the anterior ocular segment illumination positions and the split center. Next, symmetry between the positions of the reflection images resulting from the anterior ocular segment illumination is determined using the following Formula 21 by comparing the evaluation values a10 and a11 (S1301).

−1≦a11−a10≦+1 [unit: pixel] (Formula 21)

In the case where the evaluation values do not meet the conditions of Formula 21 (that is, in the case where the determination is not successful), the positions of the subject eye and the optical system are skewed in the horizontal direction, and thus the alignment is determined to be unsuccessful and the alignment determination process ends (NG in S1301; S807). On the other hand, in the case where the evaluation values meet the conditions of Formula 21 (that is, in the case where the determination is successful), the alignment determination using horizontal line pairs (S802 to S807) is executed. Note that the processing advances from S1301 to S802 in the case where the anterior ocular segment illumination is not detected.

By determining the symmetry of the reflection image positions in this manner, the reflection image positions are detected as being asymmetrical in the case of misalignment with the subject eye, and thus the success or failure of alignment can be determined quickly. Superior effects can be achieved as a result, such as reducing the load on the CPU that performs the determination, making it possible to use a lower-spec CPU, and so on. Furthermore, the determination can be performed with certainty and without failure even in the case where the anterior ocular segment illumination appears in the anterior ocular segment observation image.

Although the fifth embodiment describes executing the alignment determination described in the second embodiment in the case where the reflection image positions have been confirmed as symmetrical, the embodiment is not limited thereto, and the alignment determinations described in the first to fourth embodiments may be applied instead.

As described above, according to the ophthalmic apparatuses described in the first to fifth embodiments, alignment can be performed automatically in the anterior ocular observation state, pupil detection failures due to the influence of anterior ocular segment illumination light being reflected and so on can be reduced, and the likelihood of erroneous detection can be greatly reduced.

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (e. g., computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-247753, filed Nov. 9, 2012, which is hereby incorporated by reference herein in its entirety.

What is claimed is:

1. An alignment determination method for an ophthalmic apparatus that determines whether alignment with a subject eye is complete using an observation image of an anterior ocular segment of the subject eye obtained through a split prism, the method comprising:
a judging step of determining the success or failure of alignment based on a position of a pupil image on respective lines in a line pair that are parallel to a boundary in the split prism and are equidistant from the boundary in the observation image; and
a determining step of determining whether or not the alignment is complete based on a determination result obtained by executing the judging step for a plurality of line pairs having different distances from the boundary.

2. The method according to claim 1,
wherein in the determining step, the judging step is executed in order starting with the line pair closest to the boundary, the alignment is determined to be complete when a successful determination result is obtained, and the judging step is not executed for the remaining line pairs.

3. The method according to claim 1,
wherein in the determining step, the alignment is determined to be complete in the case where at least one of a plurality of determination results obtained by executing the judging step for all of the plurality of line pairs is successful.

4. The method according to claim 1,
wherein in the determining step, the alignment is determined to be incomplete in the case where the alignment has been determined unsuccessful for all of the plurality of line pairs.

5. The method according to claim 1,
wherein in the plurality of line pairs, the distances from the lines in a line pair to the lines in an adjacent line pair are greater than an estimated size of the reflection image resulting from the anterior ocular segment illumination.

6. The method according to claim 1,
wherein in the judging step, the success or failure of alignment is determined based on edge positions of a pupil image present on the respective lines in the line pair in the observation image and a position of a split center in the observation image.

7. The method according to claim 1, further comprising:
a step of determining that the alignment is incomplete in the case where positions of two reflection images resulting from anterior ocular segment illumination that have been detected from the observation image are asymmetrical relative to the split center.

8. The method according to claim 1, further comprising:
a step of determining whether or not the alignment is complete based on the size of a pupil image in the observation image.

9. An alignment determination method for an ophthalmic apparatus that determines whether alignment with a subject eye is complete using an observation image of an anterior ocular segment of the subject eye obtained through a split prism, the method comprising:
a detection step of detecting a reflection image resulting from anterior ocular segment illumination in the observation image;
a setting step of setting a line pair whose lines are parallel to a boundary of the split prism and that are equidistant from the boundary so that the line pair does not overlap with the reflection image in the observation image; and
a judging step of determining whether or not the alignment is complete based on positions, in the observation image, of the pupil image on the respective lines in the line pair set in the setting step.

10. The method according to claim 9,
wherein in the setting step, a single line pair is selected from a plurality of different line pairs that are prepared in advance and whose distances from the boundary are different.

11. An alignment determination method for an ophthalmic apparatus that determines whether alignment with a subject eye is complete using an observation image of an anterior ocular segment of the subject eye obtained through a split prism, the method comprising:
a calculation step of calculating respective centers of gravity for two pupil areas of a pupil image in the observation image that have been separated by a boundary of the split prism; and
a judging step of determining whether or not the alignment is complete based on the positions, in the observation image, of the two centers of gravity calculated in the calculation step and a position, in the observation image, of a split center.

12. The method according to claim 11,
wherein in the calculation step, the two centers of gravity are calculated using an area of the pupil image that is present in a range enclosed by a set of lines that are parallel to the boundary of the split prism and are equidistant from the boundary in the observation image.

13. The method according to claim 11, further comprising:
a detection step of detecting a reflection image resulting from anterior ocular segment illumination in the observation image,
wherein the calculation step and the judging step are executed in the case where the reflection image has been detected in the detection step, and in the case where the reflection image has not been detected in the detection step, the success or failure of alignment is determined based on the position of the pupil image on the respective lines of the line pair that are parallel to the boundary of the split prism and are equidistant from the boundary in the observation image.

14. An ophthalmic apparatus that determines whether alignment with a subject eye is complete using an observation image of an anterior ocular segment of the subject eye obtained through a split prism, the apparatus comprising:
a judging unit configured to determine the success or failure of alignment based on a position of a pupil image on respective lines in a line pair that are parallel to a boundary in the split prism and are equidistant from the boundary in the observation image; and
a determining unit configured to determine whether or not the alignment is complete based on a determination result from the judging unit for a plurality of line pairs having different distances from the boundary.

15. The apparatus according to claim 14, further comprising:
a unit configured to determine that the alignment is incomplete in the case where positions of two reflection images resulting from anterior ocular segment illumination that have been detected from the observation image are asymmetrical relative to the split center.

16. An ophthalmic apparatus that determines whether alignment with a subject eye is complete using an observation image of an anterior ocular segment of the subject eye obtained through a split prism, the apparatus comprising:
a detection unit configured to detect a reflection image resulting from anterior ocular segment illumination in the observation image;
a setting unit configured to set a line pair whose lines are parallel to a boundary of the split prism and that are equidistant from the boundary so that the line pair does not overlap with the reflection image in the observation image; and
a judging unit configured to determine whether or not the alignment is complete based on positions, in the observation image, of the pupil image on the respective lines in the line pair set by the setting unit.

17. An ophthalmic apparatus that determines whether or not alignment with a subject eye is complete using an observation image of an anterior ocular segment of the subject eye obtained through a split prism, the apparatus comprising:
a calculation unit configured to calculate respective centers of gravity for two pupil areas of a pupil image in the observation image that have been separated by a boundary of the split prism; and
a judging unit configured to determine whether or not the alignment with the subject eye is complete based on the positions, in the observation image, of the two centers of gravity calculated by the calculation unit and a position, in the observation image, of a split center.

18. The apparatus according to claim 17, further comprising:
a detection unit configured to detect a reflection image resulting from anterior ocular segment illumination in the observation image,
wherein the calculation unit and the judging unit determine whether alignment is complete in the case where the reflection image has been detected by the detection unit, and in the case where the reflection image has not been detected by the detection unit, the success or failure of alignment is determined based on the position of the pupil image on the respective lines of the line pair that are parallel to the boundary of the split prism and are equidistant from the boundary in the observation image.

19. A non-transitory computer-readable storage medium on which is stored a program for causing a computer to execute the steps of the alignment determination method for an ophthalmic apparatus according to claim 1.
